(19) United States
(12) Patent Application Publication
(10) Pub. No.: US 2020/0370080 A1
(43) Pub. Date: Nov. 26, 2020

(54) ENANTIONSELECTIVE ENZYMATIC SULFOXIDATION OF CHIRAL ARYLSULFIDES

(71) Applicant: Bayer Aktiengesellschaft, Leverkusen (DE)

(72) Inventors: Rainhard KOCH, Kleinmachnow (DE); Simon KLAFFL, Duesseldorf (DE); Markus SPELBERG, Duesseldorf (DE); Dirk TISCHLER, Freiberg (DE); Michael SCHLOEMANN, Freiberg (DE); Anika SCHOLTISSEK, Freiberg (DE); Thomas HEINE, Freiberg (DE); Bruno BUEHLER, Leipzig (DE); Andreas SCHMID, Dortmund (DE); Christian WILLRODT, Leipzig (DE)

(21) Appl. No.: 16/764,161

(22) PCT Filed: Jul. 12, 2018
(86) PCT No.: PCT/EP2018/068978

§ 371 Date: May 14, 2020

(30) Foreign Application Priority Data
Nov. 16, 2017 (EP) 17202022.4

(51) Int. Cl. C12P 11/00; C12N 9/02

(52) U.S. Cl. CPC C12P 11/00; C12Y 114/14011; C12Y 114/14001; C12N 9/0071

(57) ABSTRACT

What is described herein refers to isolated nucleic acid fragments encoding an oxygenase subunit (StyA) and a reductase subunit (StyB), wherein the polypeptide encoded for by the nucleotide sequence for the oxygenase subunit (StyA) and the nucleotide sequence for the reductase subunit (StyB) have activity towards chiral arylsulfides.

Chiral sulfoxides can be potentially used as buildings blocks, chiral auxiliaries or active pharmaceutical ingredients. Due to their chiral nature and the fact, that the potency of some enantiomers is higher than of their antipode (e.g. esomeprazole), asymmetric synthesis is required. This can for example be achieved via the preparative separation of the enantiomers. However, this synthesis route is time consuming and higher yields would be desirable.

Thus, it was the object of the current invention to devise an alternative more efficient and more cost effective way of providing pure enantiomers of sulfoxides.

The invention achieves this object by provision of an isolated nucleic acid fragment encoding an oxygenase subunit (StyA) and an isolated nucleic acid fragment encoding a reductase subunit (StyB), wherein the polypeptides encoded for by the nucleotide sequences for the oxygenase subunit (StyA) and the nucleotide sequence for the reductase subunit (StyB) together have activity towards chiral arylsulfides and wherein said nucleic acid fragments are selected from the group
a) nucleic acid fragment comprising a nucleotide sequence of at least 80% sequence identity to SEQ ID NO 1 and a nucleotide sequence of at least 80% sequence identity to SEQ ID NO 2
b) nucleic acid fragment comprising a sequence complementary to SEQ ID NO 1 and a nucleic acid fragment comprising a sequence complementary to SEQ ID NO 2
c) nucleic acid fragment comprising a sequence which specifically hybridizes to said nucleic acid fragment of a) or said complementary of b).

Surprisingly, it was found that the oxygenase subunit (StyA) and the reductase subunit (StyB) encoded for by the isolated nucleic acid fragments described herein together catalyze the sulfoxide formation—i.e. the oxidation of a sulfide—in chiral aryl sulfides including heteroaromatic sulfides with high activity and high enantiomeric excess.

Without wishing to be bound by theory it is assumed that the oxygenase subunit (StyA) and the reductase subunit (StyB) encoded for by the isolated nucleic acid fragments described herein represent a styrene monooxygenase.

As used herein the term “styrene monooxygenase” (used synonymously with “SMO”) refers to an enzyme that belongs to the group of E1-Typ Monooxygenase and is dependent on FAD as cofactor. Styrene monooxygenases (SMOs) are thought to be a class of enzymes performing a selective oxygenation of the vinyl side chain of styrene to styrene oxide or to oxidize indol (Sadauskas et al. 2017). Like most SMOs described so far also the polypeptides described herein form a two-component system. The two components, a single oxidase and a single FAD reductase, are encoded by two separate genes, StyA and StyB, respectively. The reductase (StyB) solely uses NADH to reduce the FAD cofactor which is then utilized by the oxidase (StyA) to activate molecular oxygen and to catalyze the oxidation.

The inventors of the current invention have shown for the first time that chiral arylsulfoxides—i.e. in this case only the S-enantiomer of the oxidized compound according to formula I—can be generated efficiently enough, enantiomerically pure enough and in suitable quantities for synthesis on a production scale using a two component SMO i.e. the polypeptides described herein. This is the case, even though styrene monooxygenase of other organism have been described previously (Tischler et al. 2009, Tischler et al. 2012).

The term “comprising” is to be interpreted as specifying the presence of the stated parts, steps or components, but does not exclude the presence of one or more additional parts, steps or components.

It is understood that when referring to a word in the singular, the plural is also included herein. Thus, reference to an element by the indefinite article “a” or “an” does not exclude the possibility that more than one of the element is present, unless the context clearly requires that there be one and only one of the elements. The indefinite article “a” or “an” thus usually means “at least one”.

In general parameters determining catalyst and process efficiency are depicted in FIG. 8.

A common unit to measure enzyme activity of whole cells is U/gcdw where U stands for unit i.e. for μmol/min. In other words if a biocatalyst has an activity of 1 U it generates 1 μmol of product per minute. As used herein the unit of enzyme activity refers to the employed biomass i.e. gramm cell dry weight (gcdw). Thus, a value of 450 U/gcdw means that 1 gramm dried cells (when the cells were still alive) catalyzed a reaction with 450 μmol/min and hence generated 450 μmol product per minute.

A reliably assertion of the suitability of a given enzyme can be made using the parameters: substrate conversion, product formation, enantiomeric excess (ee) and space time yield (STY). As used herein the term “space time yield” refers to the yield generated by a given amount of cells e.g. cultured in a culture vessel in a defined time. For example the space time yield seen in the experiments performed below was around 70 g/l in 2.3 hours.

The term “oxygenase” as used herein refers to an enzyme that oxidizes a substrate by transferring an oxygen molecule to it.

The term “reductase” as used herein refers to an enzyme which transfers electrons to an oxygenase.

The term “nucleic acid molecule” is intended to indicate any single- or double stranded nucleic acid molecule comprising DNA (cDNA and/or genomic DNA), RNA (preferably mRNA), PNA, LNA and/or Morpholino.

As used herein the term “nucleic acid fragment” refers to an isolated nucleic acid molecule.

As used herein the term “gene” means a DNA sequence made up of nucleotides comprising a region (transcribed region), which is transcribed into an RNA molecule (e.g. directly into a mRNA without intron sequences) in a cell, operably linked to regulatory regions capable of regulating the expression of the polypeptide. A gene may thus comprise several operably linked sequences, such as untranslated regulatory regions (e.g. a promoter, enhancer, repressor), a 5′ leader sequence comprising e.g. sequences involved in translation initiation, a (protein) coding region (cDNA or genomic DNA) and a 3′ non-translated sequence comprising e.g. transcription termination sites.

As used herein the term “expression of a gene” or “gene expression” refers to the process wherein a DNA region (the coding region), which is operably linked to appropriate regulatory regions, particularly a promoter, is transcribed into an mRNA molecule. The mRNA molecule is then processed further (by post-transcriptional processes) within the cell, e.g. by translation initiation and translation into an amino acid chain (polypeptide), and translation termination by translation stop codons.

As used herein the term “Wild type” (also written “wildtype” or “wild-type”), refers to a typical form of a gene as it most commonly occurs in nature.

As used herein the term “polypeptide” refers to any peptide, polypeptide, oligopeptide or protein. A polypeptide consists of consecutive amino acids, which are linked by peptide bonds. The polymer may be linear or branched, it may comprise modified amino acids, and it may be interrupted by non-amino acids. The polypeptide may be human, non-human, and an artificial or chemical mimetic of a corresponding naturally occurring amino acid, as well as naturally occurring amino acid polymers and non-naturally occurring amino acid polymers. The term also encompasses an amino acid polymer that has been modified by either natural processes or by chemical modifications; for example, by disulfide bond formation, glycosylation, lipidation, acetylation, acylation, phosphorylation, or any other manipulation, such as conjugation with a labeling component, such as but not limited to, fluorescent markers, particles, biotin, beads, proteins, radioactive labels, chemiluminescent tags, bioluminescent labels, and the like.

As used herein the term “sequence identity” of two related nucleotide or amino acid sequences, expressed as a percentage, refers to the number of positions in the two optimally aligned sequences which have identical residues (×100) divided by the number of positions compared. A gap, i.e., a position in an alignment where a residue is present in one sequence but not in the other, is regarded as a position with non-identical residues. The “optimal alignment” of two sequences is found by aligning the two sequences over the entire length. In other words if two identical sequences are aligned the sequence identity value would be 100%.

Aligned sequences of nucleotide or amino acid residues are typically represented as rows within a matrix. Gaps are inserted between the residues so that identical or similar characters are aligned in successive columns.

In order to determine the sequence identity the Needleman and Wunsch global alignment algorithm (Needleman and Wunsch, 1970, J Mol Biol 48(3):443-53) of The European Molecular Biology Open Software Suite (EMBOSS, Rice et al., 2000, Trends in Genetics 16(6): 276-277; see e.g. http://www.ebi.ac.uk/emboss/align/index.html) using default settings (gap opening penalty=10 (for nucleotides)/10 (for proteins) and gap extension penalty=0.5 (for nucleotides)/0.5 (for proteins)) can be employed. For nucleotides the default scoring matrix used is EDNAFULL and for proteins the default scoring matrix is EBLOSUM62.

As used herein the term “enantiomer” refers to one of two stereoisomers of a given molecule that are mirror images of each other that are non-superposable (not identical). A single chiral atom or similar structural feature in a compound causes that compound to have two possible structures which are non-superposable, each a mirror image of the other.

Chirality is a geometric property of some molecules and ions. A chiral molecule is non-superimposable on its mirror image. The presence of an asymmetric carbon center is one of several structural features that induce chirality in organic and inorganic molecules

As used herein the term “racemate” or “racemate substrate” refers to a mixture of two stereoisomers of a chiral molecule. In most cases a racemate has equal amounts of the two stereoisomers of a chiral molecule.

The term “complementary” as used herein refers to two nucleic acid molecules that can form specific interactions with one another. In the specific interactions, an adenine base within one strand of a nucleic acid can form two hydrogen bonds with thymine within a second nucleic acid strand when the two nucleic acid strands are in opposing polarities. Also in the specific interactions, a guanine base within one strand of a nucleic acid can form three hydrogen bonds with cytosine within a second nucleic acid strand when the two nucleic acid strands are in opposing polarities. Complementary nucleic acids as referred to herein, may further comprise modified bases wherein a modified adenine may form hydrogen bonds with a thymine or modified thymine, and a modified cytosine may form hydrogen bonds with a guanine or a modified guanine.

As used herein the term “specifically hybridize” refers to a reaction of the nucleic acid sequence in question in a hybridization solution containing 0.5 M sodium phosphate buffer, pH 7.2 containing 7% SDS, 1 mM EDTA and 100 mg/ml of salmon sperm DNA at 65° C. for 16 hours and washing twice at 65° C. for twenty minutes in a washing solution containing 9.5×SSC and 0.1% SDS.

As used herein the term “enantiomeric excess” or “ee-value” refers to a measurement for the purity of chiral substances. It reflects the degree to which a sample contains one enantiomer in greater amounts than the other. The ee value is calculated based on the masses of both enantiomers in the sample according to the following formula: ee=(mass of enantiomer 1−mass of enantiomer 2)/(mass of enantiomer 1+mass of enantiomer 2)*100. In this example enantiomer 1 is the wanted enantiomer. E.g. a sample with 70 g of enantiomer 1 and 30 g of enantiomer 2 has an ee of 40%. A racemic mixture has an ee of 0%, while a single completely pure enantiomer has an ee of 100%.

Methods for determining the ee-value are known in the art. It can e.g. be determined using gas or liquid chromatography devices, equipped with a chiral column, respectively.

When referring to the fact that an enzyme while catalyzing a reaction selectively generates the S- or the R enantiomer of a given product the terms asymmetric reaction and enantioselectivity can be used. In other words, via selective catalysis an enantiomeric excess of either the R- or the S-enantiomer is generated depending on the preference of the enzyme.

As used herein the term “chiral arylsulfide” refers to a chiral compound comprising an aryl—i.e. a functional group derived from an aromatic hydrocarbon—and a thioether.

In some embodiments the chiral arylsufide is a compound of formula I:

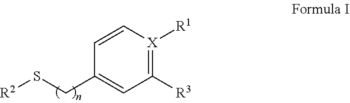

wherein X is C or N, and
wherein if X is N, R₁ is absent
and if X is C, R₁ is selected form the group consisting of a H, NO₂, a halogen, NH₂, an C₁ to C₆ alkyl, or an an C₁ to C₆ O-alkyl, and
wherein n is 0 or 1, and
wherein R² is an C₁ to C₆ alkyl and
wherein R³ is a halogen or H.

To the knowledge of the inventors the fact that the S-enantiomer of an oxidized arylsulfide comprising a halogen can be generated efficiently enough, enantiomerically pure enough and in suitable quantities for synthesis on a production scale using an enzyme has not been demonstrated before.

In some embodiments the chiral arylsufide is a compound of formula I:

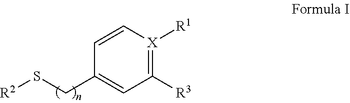

wherein X is N, and
wherein R₁ is absent
and
wherein n is 1, and
wherein R² is an C₁ to C₃ alkyl and
wherein R³ is a halogen.

In a preferred embodiment of the isolated nucleic acid fragments encoding an oxygenase subunit (StyA) and a reductase subunit (StyB) wherein the polypeptides encoded for by the nucleotide sequence for the oxygenase subunit (StyA) and the nucleotide sequence for the reductase subunit (StyB), together have activity towards chiral arylsulfides said nucleic acid fragments are selected from the group
a) nucleic acid fragment comprising a nucleotide sequence of at least 80% sequence identity to SEQ ID NO 1 and a nucleotide sequence of at least 80% sequence identity to SEQ ID NO 2
b) nucleic acid fragment comprising a sequence complementary to SEQ ID NO 1 and a nucleic acid fragment comprising a sequence complementary to SEQ ID NO 2
c) nucleic acid fragment comprising a sequence which specifically hybridizes to said nucleic acid fragment of a) or said complementary of b).
   the nucleic acid fragment comprising a nucleotide sequence with sequence identity to SEQ ID NO 1 has at least 80% sequence identity, more preferably at least 85% sequence identity, more preferably at least 90% sequence identity, more preferably at least 95% sequence identity, more preferably at least 95%, 96%, 97%, 98%, 99%, and most preferably 100% sequence identity to SEQ ID NO 1 and
   the nucleic acid fragment comprising a nucleotide sequence with sequence identity to SEQ ID NO 2 has at least 80% sequence identity, more preferably at least 85% sequence identity, more preferably at least 90% sequence identity, more preferably at least 95% sequence identity, more preferably at least 95%, 96%, 97%, 98%, 99%, and most preferably 100% sequence identity to SEQ ID NO 2.

In a further preferred embodiment, the isolated nucleic acid fragments described herein consist of
a) a nucleotide sequence of at least 80% sequence identity to SEQ ID NO 1 and a nucleotide sequence of at least 80% sequence identity to SEQ ID NO 2, respectively
b) a nucleic acid fragment comprising a sequence complementary to SEQ ID NO 1 and a nucleic acid fragment comprising a sequence complementary to SEQ ID NO 2, respectively
c) a nucleic acid fragment consisting of a sequence which specifically hybridizes to said nucleic acid fragment of a) or said complementary of b).

It is preferred that in said isolated nucleic acid fragments consisting of
a) a nucleotide sequence of at least 80% sequence identity to SEQ ID NO 1 and a nucleotide sequence of at least 80% sequence identity to SEQ ID NO 2, respectively
b) a nucleic acid fragment comprising a sequence complementary to SEQ ID NO 1 and a nucleic acid fragment comprising a sequence complementary to SEQ ID NO 2, respectively
c) a nucleic acid fragment consisting of a sequence which specifically hybridizes to said nucleic acid fragment of a) or said complementary of b)
   the nucleic acid fragment consisting of a nucleotide sequence with sequence identity to SEQ ID NO 1 has at least 80% sequence identity, more preferably at least 85% sequence identity, more preferably at least 90% sequence identity, more preferably at least 95% sequence identity, more preferably at least 95%, 96%, 97%, 98%, 99%, and most preferably 100% sequence identity to SEQ ID NO 1 and
   the nucleic acid fragment consisting of a nucleotide sequence with sequence identity to SEQ ID NO 2 has at least 80% sequence identity, more preferably at least 85% sequence identity, more preferably at least 90% sequence identity, more preferably at least 95% sequence identity, more preferably at least 95%, 96%, 97%, 98%, 99%, and most preferably 100% sequence identity to SEQ ID NO 2.

In a further aspect what is described herein relates to a recombinant expression vector comprising the isolated nucleic acid fragments described herein, wherein said recombinant expression vector is selected from the group
a) recombinant expression vector comprising the isolated nucleic acid fragments of claim 1 as well as a lac repressor comprising a nucleotide sequence of at least 80% sequence identity to SEQ ID NO 3
b) recombinant expression vector comprising a nucleotide sequence of at least 80% sequence identity to SEQ ID NO 4
c) recombinant expression vector comprising a sequence complementary to SEQ ID NO 4
d) recombinant expression vector comprising a sequence which specifically hybridizes to said nucleic acid fragment of b) or said complementary of c).

Surprisingly it was found that the isolated nucleic acid fragment described herein is expressed especially well from a vector comprising the lac repressor sequence.

The term “vector” or “recombinant expression vector” or “expression vector”, as used herein, refers to a molecular vehicle used to transfer foreign genetic material into a cell. The vector itself is generally a DNA sequence that consists of an insert (sequence of interest) and a larger sequence that serves as the “backbone” of the vector. The purpose of a vector to transfer genetic information to another cell is typically to isolate, multiply, or express the insert in the target cell.

It is known in the art that instead of using one expression vector for gene expression two or more expression vectors can be employed or that isolated nucleic acid fragments to be expressed in a given host cell can be inserted into the genome of said cell. Moreover, combinations of these methods can be used.

In a further preferred embodiment the recombinant expression vector comprising the isolated nucleic acid fragments described herein is selected from the group
a) recombinant expression vector comprising the isolated nucleic acid fragments described above as well as a lac repressor comprising a nucleotide sequence of at least 80% sequence identity, more preferably at least 85% sequence identity, more preferably at least 90% sequence identity, more preferably at least 95% sequence identity, more preferably at least 95%, 96%, 97%, 98%, 99%, and most preferably 100% sequence identity to SEQ ID NO 3,
b) recombinant expression vector comprising a nucleotide sequence of at least 80% sequence identity, more preferably at least 85% sequence identity, more preferably at least 90% sequence identity, more preferably at least 95% sequence identity, more preferably at least 95%, 96%, 97%, 98%, 99%, and most preferably 100% sequence identity to SEQ ID NO 4
c) recombinant expression vector comprising a sequence complementary to SEQ ID NO 4
d) recombinant expression vector comprising a sequence which specifically hybridizes to said nucleic acid fragment of b) or said complementary of c).

In a preferred embodiment of the isolated nucleic acid fragments described herein, the nucleotide sequence for the oxygenase subunit (StyA) codes for a polypeptide with at least 80% sequence identity, more preferably at least 85% sequence identity, more preferably at least 90% sequence identity, more preferably at least 95% sequence identity, more preferably at least 95%, 96%, 97%, 98%, 99%, and most preferably 100% sequence identity to SEQ ID NO 5 and wherein the nucleotide sequence for the reductase subunit (StyB) codes for a polypeptide with at least 80% sequence identity, more preferably at least 85% sequence identity, more preferably at least 90% sequence identity, more preferably at least 95% sequence identity, more preferably at least 95%, 96%, 97%, 98%, 99%, and most preferably 100% sequence identity to SEQ ID NO 6.

A skilled person is aware that the polypeptides characterized herein via their amino acid sequence, can be encoded for by different nucleic acids sequences. This is a result of the degeneracy of the genetic code

As a result of this degeneracy of the genetic code, amino acids can be encoded by one or more codons. In different organisms, the codons coding for an amino acid are used at different frequencies. Adapting the codons of a coding nucleic acid sequence to the frequency of their use in the organism in which the sequence to be expressed is to be integrated may contribute to an increased amount of translated protein and/or to the stability of the mRNA in question in the particular plant cells or plants. The frequency of use of codons in the host cells or hosts in question can be determined by the person skilled in the art by examining as many coding nucleic acid sequences of the organism in question as possible in terms of the frequency with which certain codons are used for coding a certain amino acid. The frequency of the use of codons of certain organisms is known to the person skilled in the art and can be determined in a simple and rapid manner using specifically developed algorithms implemented into computer programs (e.g. Grote et al., 2005, Nucleic Acids Research 33, W526-W531; doi: 10.1093/nar/gki376). Tools using such algorithms are publicly accessible and are provided for free as web-interfaces inter alia on the World Wide Web from various institutions, like the European Bioinformatics Institute (EMBL-EBI) and others (for example http://www.jcat.de; http://gcua.schoedl.de/; http://www.kazusa.or.jp/codon/; http://www.entelechon.com/eng/cutanalysis.html; http://www.ebi.ac.uk/Tools/st/emboss_backtranseq/). Adapting the codons of a coding nucleic acid sequence to the frequency of their use in an organism in which the sequence is intended to be expressed can be carried out by in vitro mutagenesis or, preferably, by de novo synthesis of the gene sequence. Methods for the de novo synthesis of nucleic acid sequences are known to the person skilled in the art. A de novo synthesis can be carried out, for example, by initially synthesizing individual nucleic acid oligonucleotides, hybridizing these with oligonucleotides complementary thereto, so that they form a DNA double strand, and then ligating the individual double-stranded oligonucleotides such that the desired nucleic acid sequence is obtained. The de novo synthesis of nucleic acid sequences including the adaptation of the frequency with which the codons are used to a certain target organism can also be sourced out to companies offering this service (for example Eurofins MWG).

In some embodiments the isolated nucleic acid fragments described above are characterized in the nucleotide sequence for the oxygenase subunit (StyA) codes for the polypeptide of SEQ ID NO 5 and wherein the nucleotide sequence for the reductase subunit (StyB) codes for the polypeptide of SEQ ID NO 6.

In a further aspect what is described herein relates to the use of the oxygenase subunit (StyA) described herein together with the reductase subunit (StyB) described herein for the enantioselective oxidation of a compound according to formula I

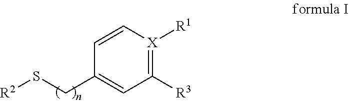

wherein X is C or N, and
wherein if X is N, R₁ is absent
and if X is C, R₁ is selected form the group consisting of a H, NO₂, a halogen, NH₂, an C₁ to C₆ alkyl, or an an C₁ to C₆ O-alkyl, and
wherein n is 0 or 1, and
wherein R² is an C₁ to C₆ alkyl and
wherein R³ is a halogen or H
into the S-sulfynil-enantiomer.

As used herein the term “alkyl” refers to a linear or branched, substituted or unsubstituted, saturated or unsaturated or but not cyclic hydrocarbon chain.

In a preferred embodiment, the alkyl is a linear, unsubstituted, saturated hydrocarbon chain.

In an especially preferred embodiment the alkyl is an ethyl or methyl group.

In a further preferred embodiment R₁ is selected form the group consisting of H, NO₂, a halogen, an C₁ to C₆ alkyl, or an an C₁ to C₆ O-alkyl.

Preferably, the halogen is selected from fluor, bromide or chloride,

In some embodiments of the use the oxygenase subunit (StyA) described herein together with the reductase subunit (StyB) described herein are used for the enantioselective oxidation of a compound according to formula I,

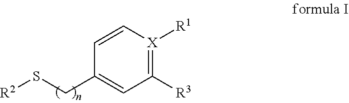

wherein X is N, and
wherein R₁ is absent
and
wherein n is 1, and
wherein R² is an C₁ to C₃ alkyl and
wherein R³ is a halogen
into the S-sulfynil-enantiomer.

In one embodiment the compound of formula I is selected from the group consisting of phenyl methyl sulfide, 4-methylphenyl methyl sulfide, 4-fluorophenyl methyl sulfide, 4-chlorophenyl methyl sulfide, 4-bromophenyl methyl sulfide, 4-methoxyphenyl methyl sulfide, 4-nitrophenyl methyl sulfide, ethyl phenyl sulfide, benzyl methyl sulfide, phenyl vinyl sulfide, 2-chloro-4-(methylsulfanylmethyl)pyridine.

In an especially preferred embodiment the compound of formula I is 2-chloro-4-(methylsulfanylmethyl)pyridine.

In some embodiments an oxygenase subunit (StyA) with 80% sequence identity to SEQ ID NO 5 together with an reductase subunit (StyB) with 80% sequence identity to SEQ ID NO 6 is employed for the use described above.

In some embodiments an oxygenase subunit (StyA) with 80% sequence identity to SEQ ID NO 5 together with the reductase subunit (StyB) of SEQ ID NO 6 is employed for the use described above.

In some embodiments the oxygenase subunit (StyA) of SEQ ID NO 5 together with an reductase subunit (StyB) with 80% sequence identity to SEQ ID NO 6 is employed for the use described above.

In some embodiments the oxygenase subunit (StyA) of SEQ ID NO 5 together with the reductase subunit (StyB) of SEQ ID NO 6 is employed for the use described above.

In a further aspect what is described herein relates to a method for the enantioselective oxidation of a compound according to formula I into the S-sulfynil-enantiomer comprising
providing said compound according to formula I,
providing the oxygenase subunit (StyA) described herein together with the reductase subunit (StyB) described herein as polypeptides,
reacting said compound according to formula I with said polypeptides for 2-48 hours at a pH of 5-9 and a temperature of 15-40° C.

This method allows the enantioselective generation of the S-enantiomer of the oxidized compound according to formula I with a high yield in a short time period (cf. FIG. 7). In other words, it was surprisingly found that the oxygenase subunit (StyA) described herein together with the reductase subunit (StyB) described herein through oxidizing the compound according to formula I selectively generate the S-enantiomer.

As stated above the reaction is carried out for 0.5-48 hours at a pH of 5-9 and a temperature of 15-40° C., preferably for 1-24 hours at a pH of 6-8 and a temperature of 25-35° C., most preferably for 1-4 hours at a pH of 7.2 and a temperature of 30° C.

The method described herein can be carried out via providing the polypeptides described herein as mixture of the purified oxygenase subunit (StyA) and the purified reductase subunit (StyB), as cell-free enzyme extract or through providing whole cells expressing both the oxygenase subunit (StyA) and reductase subunit (StyB).

In a preferred embodiment of the method described herein, the method further comprises the step of providing the S-enantiomer of the oxidized compound according to formula I, i.e. purifying said S-enantiomer of the oxidized compound according to formula I.

In an embodiment of the method described herein, the oxygenase subunit (StyA) described herein and the reductase subunit (StyB) described herein are expressed in recombinant cells provided in a cell culture medium and the compound according to formula I is directly added to the cell culture.

This method employing whole-cell biocatalysis has the advantage that the product i.e. S-enantiomer of the oxidized compound according to formula I was generated in very high quantities (cf. FIG. 3 and FIG. 4) significantly exceeding production rates of whole cell biocatalysts reactions described so far (cf. FIG. 5). Moreover, as shown below the achieved specific activity and the resulting STY in the whole cell biocatalysis system was several orders of magnitude higher than the use of cell lysate.

| | Cofactor | Further | | |
|---|---|---|---|---|
| Conditions | recycling | additives | Specific activity | |
| Cell free | GDH based | NAD, FAD, | 0.7 | U/g CDW |
| extract | system | Glucose | | |
| Whole cell | Whole cells | | 450 | U/g CDW |
| system | | | | |

For cofactor dependent enzymes such as the oxygenase subunit (StyA) described herein another advantage of catalysis reaction in whole cells is the ability of the microbial metabolism for in vivo cofactor regeneration. This is especially an advantage as stoichiometric addition of the cofactor NAD(P)H is not economically.

Although whole-cell bioprocesses have been described for oxyfunctionalizations (Schrewe et al., 2013), the inventors of the current invention have shown for the first time that chiral arysulfoxides—i.e. in this case only the S-enantiomer of the oxidized compound according to formula I—can be generated efficiently enough, enantiomerically pure enough and in suitable quantities for synthesis on a production scale using an SMO—i.e. the polypeptides described herein—as whole cell biocatalysts.

As used herein the term “biocatalysis” refers to a reaction catalyzed by enzymes.

As used herein the term “whole-cell biocatalysis” refers to a catalysis in living cells by enzymes that said are recombinantly expressed by said cells.

In a preferred embodiment of the method employing whole-cell biocatalysis described herein, the oxygenase subunit (StyA) described herein and the reductase subunit (StyB) described herein are expressed in recombinant cells provided in a cell culture medium, the compound according to formula I is directly added to the cell culture medium once the cells have reached a predetermined cell density and wherein the product, i.e. the S-enantiomer of the oxidized compound according to formula I accumulates in the cell culture medium.

The term “cell viability” as used herein refers to the ability of cells in culture to survive under a given set of culture conditions or experimental variations. The term as used herein also refers to that portion of cells which are alive at a particular time in relation to the total number of cells, living and dead, in the culture at that time.

The term “cell density” as used herein refers to that number of cells present in a given volume of medium.

A person skilled in the art knows how to determine the cell viability and the cell density. One example of measuring cell density is via measuring absorbance, or optical density, of a sample at a wavelength of 600 nm. It is a common method for estimating the concentration of bacterial or other cells in a liquid. Measuring the concentration can indicate the stage of cultured cell population, i.e. whether it is in lag phase, exponential phase, or stationary phase.

In one embodiment of said aspect the employed recombinant cells are selected from the group consisting of E. coli, Pseudomonas, Corynebacterium, Bacillus, Saccharomyces, Pichia and the wildtype strain A. baylyi.

In a preferred embodiment of said aspect the employed recombinant cells are E. coli selected from the group comprising of JM 101, MG1655 and W3110, DH5α, DH10B; JM109, BL21(DE3).

In one embodiment of said aspect the recombinant cells expressing the oxygenase subunit (StyA) described herein and the reductase subunit (StyB) described herein, i.e. the styrene monooxygenase from A. baylyi, are cultured until a predetermined cell density is reached, then the cells are harvested, washed and returned to the culture vessel. Thus, the subsequent biocatalysis is carried out in living cells which have stopped growing.

Additionally or alternatively none or too little MgSO₄ is added to the cell culture medium. Hence, the cells stop growing once the MgSO₄ has been consumed. Other limitations can be introduced via limiting the supply of nitrogen, phosphate and/or sulphur.

Thus, in a preferred embodiment of said aspect the recombinant cells expressing the oxygenase subunit (StyA) described herein and the reductase subunit (StyB) described herein, are grown until a cell density of 0.5-2 is reached at OD 600, preferably until a cell density of 0.6 is reached at OD 600 and then the cells are harvested and washed and/or Mg supply is limited.

In a preferred embodiment of the method described herein the compound according to formula I is added continuously or at regular time intervals at a concentration, which is below the conversion rate of the oxygenase subunit (StyA) and the reductase subunit (StyB) described herein.

In other words, the concentration of the compound according to formula I is lower than the maximum rate at which the oxygenase subunit (StyA) and the reductase subnit (StyB) can process the compound according to formula I. Hence, before the compound according to formula I can accumulate it is already converted to the product. This is especially advantageous, if the compound according to formula I is toxic to the cells at higher concentrations.

A person skilled in the art knows how to determine the maximum rate at which the enzyme can process the substrate and thus how much substrate can be added to a given reaction continuously or in a given time interval. To understand the toxicity of educt and product the growth rate of the production strain is determined in presence of different educt and product concentration.

For example in the case of the enantioselectve oxidation of 2-chloro-4-(methylsulfanylmethyl)pyridine to 2-Chloro-4-((methyl-S-sulfinyl)methyl)pyridine in E. coli cells using the oxygenase subunit (StyA) described herein and the reductase subunit (StyB) described herein, it was found that a substrate concentration above 10 mM entirely inhibited the growth of the cells. The toxicity of the product was significantly lower.

Thus the amount of educt, which is added per time interval is determined by the toxicity of the educt and the maximum conversion rate of the cells. By way of example cells with a total activity of 450 U/gcwd (cell weight dry) are used. For cells of this activity the maximum rate at which potentially toxic educt can be added without harming the cells would in theory thus be around 450 μmol/min. To be on the safe side a feeding rate which corresponds to a conversion rate of 150 U/gcdw is hence usually employed. However, the feeding rate can be raised to a level corresponding to an activity of 300 U/ gcdw without harming the cells. As the activity of the cells in this example can also be higher up to 600 U/gcdw and lower (150 U/gcdw). The feed rate has to be adapted accordingly.

The very high activity of the oxygenase subunit (StyA) described herein and the reductase subunit (StyB) described herein at low substrate concentrations is the prerequisite for the possibility to add the compound according to formula I continuously or in regular time intervals.

The whole cell biocatalysis can be carried out in aqueous medium—also termed “one phase system”—or in a combination of aqueous and organic media—also termed “two phase system”. Organic media have been developed for biocatalysis as a consequence of the poor solubility in aqueous media of many organic compounds of commercial interest which can potentially be transformed by enzymes or microorganisms. The advantages of aqueous/apolar medium two-phase systems, however, include not only the production of sparingly water-soluble compounds, but also the maintenance of a low concentration of toxic or inhibitory compounds in the aqueous phase. Typical organic phases are: DEHP: Di(2-ethylhexyl)phthalat or Bis(2-ethylhexyl)phthalate, DINP (Diisononylphthalat), DBP (Dibutylphthalat) and DIBP (Diisobutylphthalat). The following second phases can be used as well: medium and long chain alkans (C10-C16), long chain alkylic alcohols (C12-C14), esters of long chain fatty acids such as ethyloleat, butyloleat, butylstearate, alcohols of unsaturated fatty acids (oleyalkohol), long chain fatty acids (C10-C12), oleic acid, silicon oil, olive oil and corn oil.

In general there are several options to further optimize the whole cell biocatalysis described above.

For example the biocatalyst reaction can be carried out in cells showing an even higher expression rate of the polypeptides described herein. This higher expression rate leads to a higher enzymatic activity and therefore higher yields. In order to protect the biocatalyst reaction from the influence of highly reactive oxygen species produced in those high yielding cells additional detoxifying enzymes such as catalase, superoxyd dismutase or glutathion peroxydase can beco expressed with the polypeptides described herein.

Moreover, depending on the interaction of the polypeptides described herein and the metabolism of the host cell it can also be expedient to adapt the expression level in order to achieve a well balance conversion rate of all contributing enzymes.

Therefore alternatively or in addition, a higher STY is possible in case of a soluble or volatile product, via continuously removing the potentially toxic product. This can be achieved for example through the use of a continuous culture plus cell retention or a permanent binding of the product to a resin. In case of a volatile product said volatile product can be removed via the gas phase.

In a further aspect what is described herein refers to the use of the method described herein to provide the S-enantiomer of the oxidized compound according to formula I with an ee-value of more than 95%.

It was surprisingly found that in addition to its remarkable high activity the oxygenase subunit (StyA) described herein and the reductase subunit (StyB) described herein generate the S-enantiomer of the oxidized compound according to formula I with remarkably enantiomeric excess rates e.g. of more than 95% and partly even more than 99% both in vitro and in vivo (cf. FIG. 6 and FIG. 7).

This is an important finding, as typically for pharmaceutical agents, a minimum enantiomeric purity of 98% enantiomeric excess (ee) is a prerequisite and enantiomeric ratios above 98% are difficult to obtain both enzymatically and via chemical synthesis.

Moreover, this result was also unexpected since as mentioned above all styrene monooxygenases described so far have a very limited substrate tolerance.

In another preferred embodiment of said use the S-enantiomer of the oxidized compound according to formula I is generated at a rate of 10 to 60 g/l×h, even more preferably at a rate of 20 to 50 g/l×h and most preferably at a rate of 30-40 g/l×h.

In an especially preferred embodiment of said use 2-chloro-4-((methyl-S-sulfinyl)methyl)pyridine is generated enantioselective from 2-chloro-4-(methylsulfanylmethyl)pyridine at a rate of 30-40 g/l×h.

Thus, the selective oxidation to only the S-enantiomer of the compounds according to formula I in combination with its high activity renders the polypeptides described herein very interesting for a wide variety of applications in synthesis of chiral sulfoxides.

In addition, the use of enzymes in chemical synthesis usually reduces waste.

Taken together these findings render the polypeptides described herein suitable for synthesis on a production scale.

### FIGURES

FIG. 1 shows a schematic overview of the reaction of one embodiment of the invention. In this case the substrate is 4-chlorophenyl methyl sulfide. The substrate is added to a cell culture, the reaction takes place inside the cells and the reaction product accumulates in the cell culture medium.

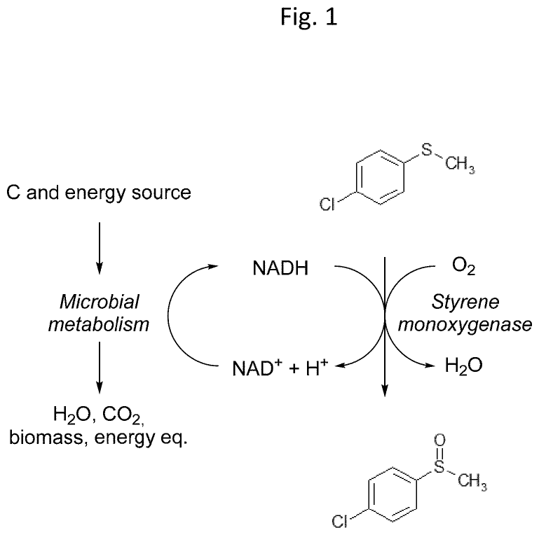

FIG. 2 depicts a schematic illustration of the employed pStyAB_ADP1_lac vector.

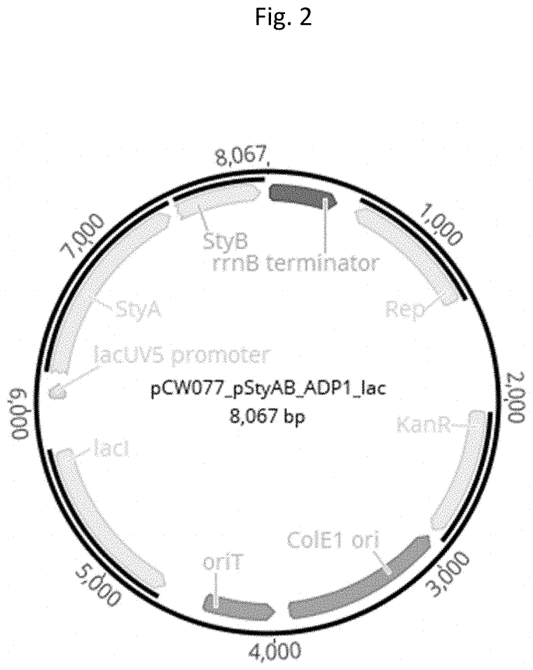

FIG. 3 shows the specific activity of the styrene monoxygenase from A. baylyi expressed from the pStyABP_ADP1_lac vector (squares) and microbial growth (diamonds) in dependency of the cultivation time.

FIG. 4 The graph of FIG. 4 demonstrates that product accumulated to very high concentrations exceeding 62 g L−1 after 2.3 h of biotransformation. No substrate accumulation was observed within the investigated time period

FIG. 5: The table of FIG. 5 demonstrates that the maximal specific production rate of the whole cell biocatalysis was surprisingly considerably higher than it could be expected from the literature.

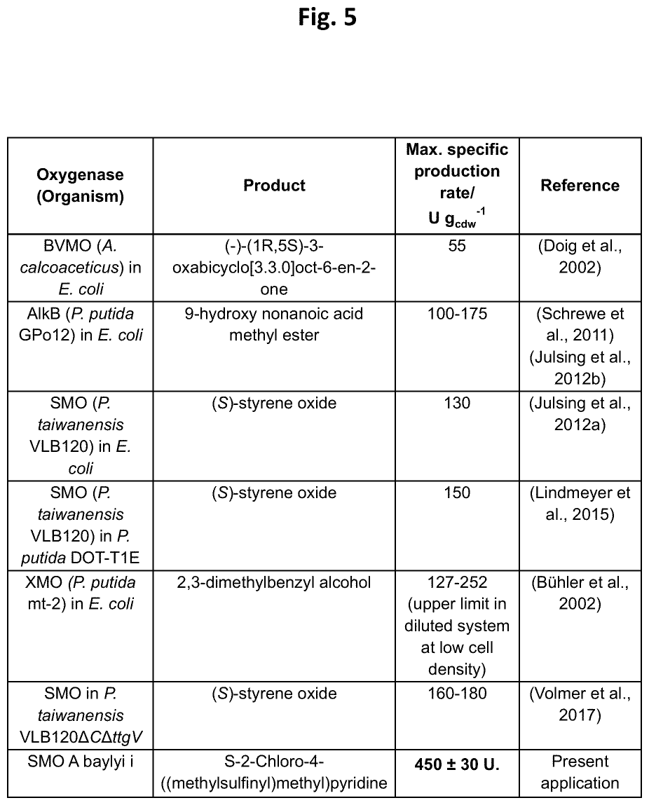

FIG. 6 The Table of FIG. 6 demonstrates that the styrene monooxygenase A. baylyi described herein converts a variety of different substrates in vitro.

FIG. 7 The Table of FIG. 7 demonstrates that the styrene monooxygenase of A. baylyi described herein converts a variety of different substrates in vivo.

FIG. 8 shows the key parameters for the characterization and quantification of cell-free and whole-cell processes as published by Schrewe et al. 2013.

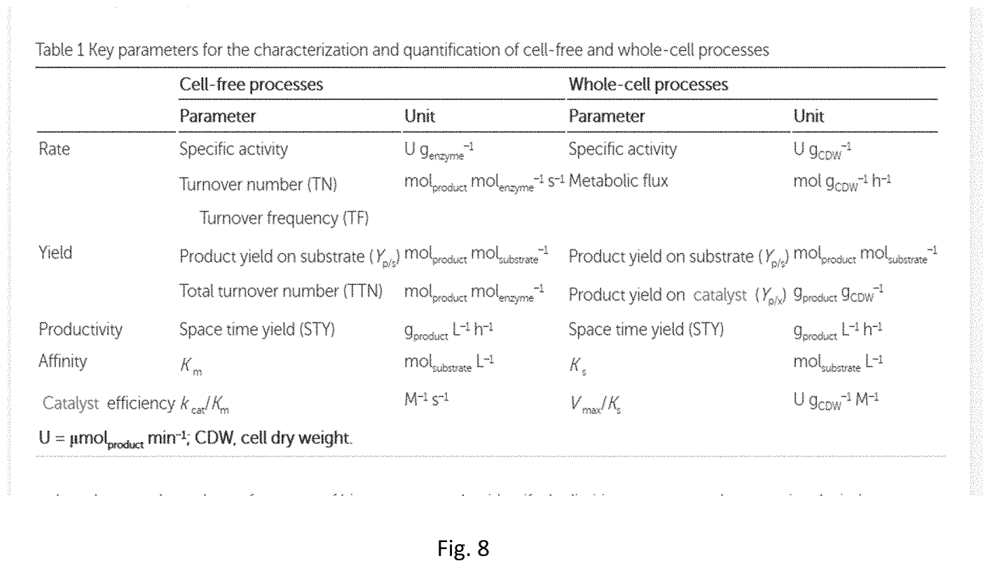

### EXAMPLES

The following examples are illustrative and not limiting. One of skill will recognize a variety of non-critical parameters that can be altered to achieve essentially similar results.

### 1) Identification and Characterization of a Novel Styrene Monooxgenase

Gene sequences of known SMOs and monooxygenases were screened for promoter and or ribosome entry sites in order to detect potentially functional enzymes. In the next step the different SMOs were expressed e.g. in E. coli. Moreover, structural models of the different SMOs were created and the different SMOs were screened for activity with the potential substrate 2-chloro-4-(methylsulfanylmethyl)pyridine. Surprisingly, it was found that the isolated nucleic acid fragments encoding an oxygenase subunit (StyA) and a reductase subunit (StyB) respectively described herein was able to generate the (S)-enantiomer of -2-chloro-4-(methylsulfinylmethyl)pyridine in high enantiomeric excess.

In detail, the isolated nucleic acid fragments encoding an oxygenase subunit (StyA) and a reductase subunit (StyB), respectively described herein from A. baylyi were expressed and it was demonstrated that chiral sulfoxides are generated using each of the substrates given in the table below. In order to do so, the StyA unit from Acinetobacter sp. ADP1 was cloned into a pET-vector inducible with IPTG and expressed in E. coli BL21 pLysS grown in LB medium (10 g/l tryptone, 5 g/l yeast extract, 10 g/l or 20 g/l NaCl, 100 μg/ml ampicillin, 50 μg/ml Chloramphenicol). For the expression a 5 ml overnight culture was used as inoculum for a fermenter with 31 medium or a flask with 500 ml medium. The cells were cultured at 37° C. until the culture reached an optical density of 0.6 which was the threshold for induction of recombinant gene expression using IPTG (0.1 mM). The culture was performed for another 18 h at 20° C. During this time the medium turned blue, if the expression was successful due to indigo formation. The cells were harvested and the cellular walls were disrupted using a French press. The protein was located in the soluble part of the cell extract, which was separated via centrifugation (45 min at >20000×g). The target protein was purified for the subsequent characterization.

The StyB unit from Acinetobacter baylyi sp. ADP1 was expressed in a similar fashion.

In order to test the activity the StyB unit (reductase) was employed in excess to supply the StyA unit (oxygenase) with sufficient reduced FAD. Therefore, NADH was produced by a format-dehydrogenase from format and NAD in the respective enzymatic reaction.

The enzyme conversion rate was probed every minute for 15 min for the kinetic analysis. If possible, i.e. soluble, 2 mM substrate were employed. In case of clouding 1 mM substrate was employed. The substrate was dissolved in ethanol. Reactions in the probes were stopped with a 1:1 mixture of ice-cold methanol and acetonitrile and afterwards analyzed using HPLC.

The educt and the product were analyzed on an Agilent 1100 HPLC device.

The results are shown in the table below:

| Substrat | yield %) | ee % | Abs. confi. |
|---|---|---|---|
| Ph—S—CH3 | 48 | 99.3 | S |
| p(Cl)—Ph—S—CH3 | 34 | 95.5 | S |
| p(Br)—Ph—S—CH3 | 58 | 98.1 | S |
| p(F)—Ph—S—CH3 | 55 | 99.0 | S |
| p(CH3)—Ph—S—CH3 | 42 | 97.6 | S |
| Ph—S—CH═CH2 | 40 | 99.7 | S |

### 2) Whole Cell Biocatalysis Using the Novel SMO

In order to further increase production rates it was tried to generate the product, i.e. the S-enantiomer of -2-chloro-4-(methylsulfinylmethyl)pyridine using a whole cell bio-catalysis in a minimal medium employing live cells even though the substrate is potentially toxic. Ideally, slowly growing or no-growing cells, i.e. resting but metabolically active cells, were used. To further enhance the enantiomeric excess (ee) value a novel vector, the pCom10:lac Vector, was developed.

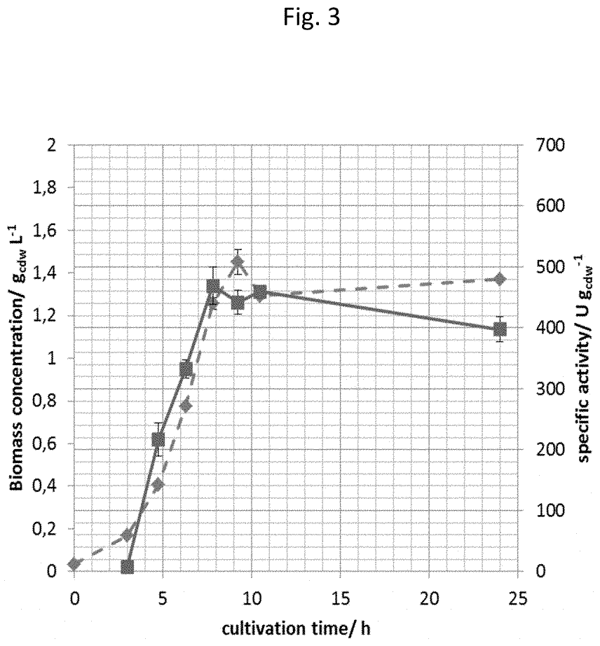

In detail, the styrene monooxygenase subunits StyA and StyB were expressed in E. coli JM101 cultivated in M9 minimal medium from the pStyAB_ADP1_lac vector. To find the maximum specific activity samples were taken after previously defined time points after addition of the inducer (1 mM IPTG). Cells were retrieved from the actively growing culture and used for the determination of the resting cell activity. As shown in FIG. 3 the specific activity increased steadily from the time point of induction and reached a maximum of 450±30 U/gcdw 4.8 h after induction.

The success of the biotransformation was assessed based on substrate conversion, product formation, and enantiomeric excess (ee) determined by chiral HPLC and GC.

In detail, the specific activity was quantified as follows: StyA and StyB were expressed in E. coli JM101 carrying the pStyAB_ADP1_lac vector. Cells were cultivated in M9 minimal medium and induced at an OD of 0.6-0.8. After 3-5 hours the cell were harvested, washed in PP buffer and resuspended in the same buffer containing glucose Then 750 μl of cells (diluted if needed) were taken and incubated in 2 ml tubes on a thermomixer set to 30° C. and 1500 rpm. Cell suspensions were pre-warmed for 5 min and then 2 mM of the substrate was added from 100-fold concentrated isopropanol stock solutions. After desired time points, 40 μL of 20% (w/v) perchloric acid was added to quench the reaction. The whole-cell biocatalyst is inactivated directly upon addition of perchloric acid that leads to a pH shift from pH 7.4 to 2.0 The addition of perchloric acid had no effect on the product itself, however, the protein immediately precipitates and was separated via centrifugation.

Successful separation of the product i.e. the S-enantiomer of 2-chloro-4-(methylsulfinylmethyl)pyridine was achieved on a Dionex UltiMate 3000 HPLC system (Thermo Scientific) equipped with a non-polar HPLC column with C18 matrix (Accucore C18, 3×150 mm, 2.6 μm particle size, Thermo Scientific). The oven was set to 30° C. and the analytes were eluted isocratically (0-6 min) 80% 10 mM ammonium phosphate buffer and 20% ACN and a subsequent (6-18 min) gradient to 90% ACN. Afterwards the column was re-equilibrated 2 min at 80% 10 mM ammonium phosphate buffer and 20% ACN. 2 μL of the respective sample were injected. The organic compounds were detected by absorption using diode array detector (DAD) at a wavelength of 210 nm. The sample preparation for analytical standards was performed as follows: 5 μL of 100-fold concentrated isopropanol stock solutions of educt or product were added to 495 μL potassium phosphate buffer (pH 7.4, 0.1 M) and subsequently 500 μL of a 1 to 1 (v/v) mixture of ACN and MeOH were added. The sample was centrifuged after mixing and directly subjected to HPLC analysis.

In the next step a preparation on technical scale was conducted.

A technical scale bioreactor (3 L total volume) experiment was performed to produce the product on a larger scale. For this, E. coli JM101 (pStyAB_ADP1_lac) was cultivated in a 3 L stirred tank reactor equipped with two Rushton impellers. The working volume was set to 2 L. The cells were cultivated in M9 minimal medium in batch mode overnight (12 h, 1.5% (w/v) glucose) followed by a glucose limited fed-batch phase (6 h). The pH was controlled at 7.2 by titration with 15% phosphoric acid and 25% (v/v) ammonium hydroxide. The gene expression of the subunits StyA and StyB was induced 1 h after start of the fed batch phase with IPTG. The growth rate was set to ˜0.18 h¹ in order prevent acetate formation and to reach a cell density of approximately 20 gcdw L⁻¹ after 6 h of glucose feed. Subsequently, the cells were harvested by centrifugation and resuspended in 2 L 0.1 M potassium phosphate buffer (pH 7.4). The specific activity of the whole cell biocatalyst was determined in separate resting cell assays. Only 1.5% glucose was added to the bioreactor to provide the living cells with energy. A constant educt feed (˜0.82 g min−1) i.e. a feed with educt (2-chloro-4-(methylsulfanylmethyl)pyridine was started 5 min later. This educt feed ensured that the available specific activity of the whole cell biocatalyst was so high that the added educt was immediately converted into the less toxic product product i.e. the S-enantiomer of -2-chloro-4-(methylsulfinylmethyl)pyridine. Samples for biomass, product/substrate and glucose/acetate were retrieved every 15 minutes.

This approach led to an efficient product formation without detectable substrate accumulation until the solubility of the product was exceeded.

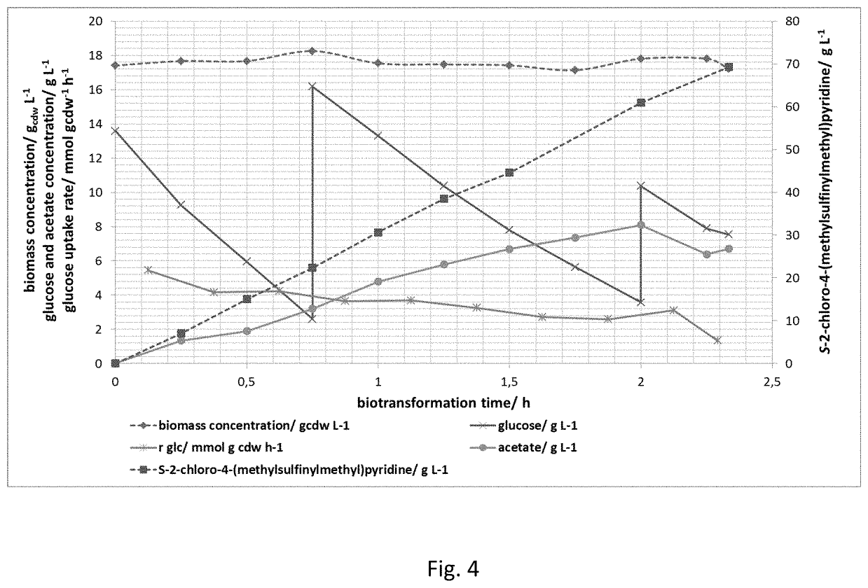

As shown in FIG. 4 product accumulated to surprisingly high concentrations exceeding 62 g L⁻¹ after 2.3 h, i.e. 30 g L⁻¹ per hour of biotransformation. No substrate accumulation was observed within the investigated time period. Product was analyzed as described above.

3) Variety of Substrates that can be Employed

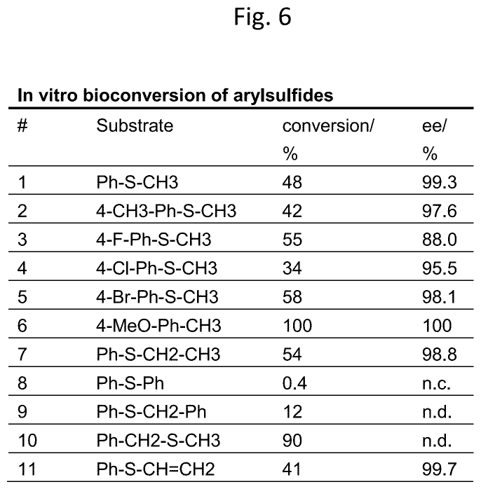

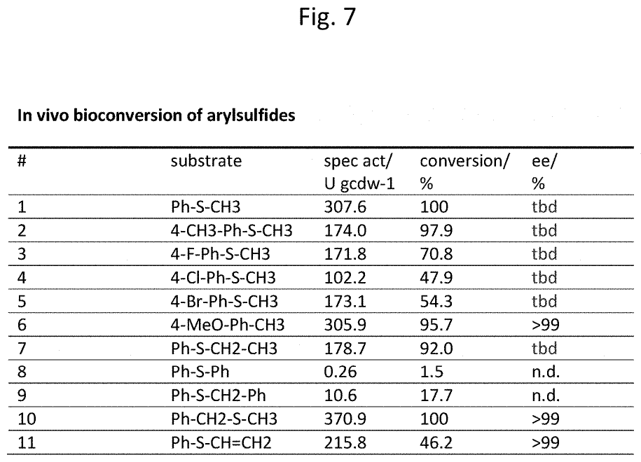

In addition it was surprisingly found that the polypeptides described herein does not only accept 2-chloro-4-(methylsulfanylmethyl)pyridine as substrate, but converts a variety of different substrates with excellent enantioselectivity. The observed efficiency of the biocatalyst with respect to activity and extremely high ee values was unexpected, since the biocatalysts that have been shown to form enantiopure sulfoxides from the corresponding sulfides so far were strongly dependent on the arylsulfide applied (Rioz-Martínez et al., 2010, Adam et al., 2005). In detail, the results presented in FIG. 6 and FIG. 7 demonstrate that the claimed enzyme converts a variety of different substrates with excellent enantioselectivity both in vitro and in vivo using biotransformation.

### REFERENCES

Adam W, Heckel F, Saha-mo C R, Taupp M, Meyer J, Schreier P. 2005. Opposite Enantioselectivities of Two Phenotypically and Genotypically Similar Strains of Pseudomonas frederiksbergensis in Bacterial 71:2199-2202.
Bühler B, Witholt B, Hauer B, Schmid A. 2002. Characterization and application of xylene monooxygenase for multistep biocatalysis. Appl. Environ. Microbiol. 68:560-568.
Doig S D, Avenell P J, Bird P A, Gallati P, Lander K S, Lye G J, Wohlgemuth R, Woodley J M. 2002. Reactor operation and scale-up of whole cell Baeyer-Villiger catalyzed lactone synthesis. Biotechnol. Prog. 18:1039-1046.
Julsing M K, Kuhn D, Schmid A, Bühler B. 2012a. Resting cells of recombinant E. coli show high epoxidation yields on energy source and high sensitivity to product inhibition. Biotechnol. Bioeng. 109:1109-1119.
Julsing M K, Schrewe M, Cornelissen S, Hermann I, Schmid A, Büler B. 2012b. Outer membrane protein AlkL boosts biocatalytic oxyfunctionalization of hydrophobic substrates in Escherichia coli. Appl. Environ. Microbiol. 78:5724-5733.
Lindmeyer M, Meyer D, Kuhn D, Bühler B, Schmid A. 2015. Making variability less variable: matching expression system and host for oxygenase-based biotransformations. J. Ind. Microbiol. Biotechnol. 42:851-866.
Rioz-Martínez A, De Gonzalo G, Pazmiño D E T, Fraaije M W, Gotor V. 2010. Enzymatic synthesis of novel chiral sulfoxides employing Baeyer-villiger monooxygenases. European J. Org.
Sadauskas, M., Vaitekūnas, J., Gasparavičiūtė, R., Mešksy, R. (2017) Genetic and Biochemical Characterization of Indole Biodegradation in Acinetobacter sp. Strain O153 Appl. Environ. Microbiol. doi:10.1128/AEM.01453-17
Schrewe M, Julsing M K, Bühler B, Schmid A. 2013. Whole-cell biocatalysis for selective and productive C—O functional group introduction and modification. Chem. Soc. Rev. 42:6346-77
Schrewe M, Magnusson A O, Willrodt C, Bühler B, Schmid A. 2011. Kinetic Analysis of Terminal and Unactivated C□H Bond Oxyfunctionalization in Fatty Acid Methyl Esters by Monooxygenase-Based Whole-Cell Biocatalysis. Adv. Synth. Catal. 353:3485-3495.
Tischler, D., Eulberg, D., Lakner, S., Kaschabek S., van Berkel W., Schlömann, M. (2009) Identification of a Novel Self-Sufficient Styrene Monooxygenase from Roodococcus opacus 1CP, JOURNAL OF BACTERIOLOGY, August 2009, p. 4996-5009
Tischler D, Gröning J A D, Kaschabek S R, Schlömann, M. (2012) One-component 690 styrene monooxygenase: an evolutionary view on a rare class of flavoproteins. Appl. 691 Biochem. Biotechnol. 167:931-944
Volmer J, Schmid A, Bühler B. 2017. The application of constitutively solvent-tolerant P. taiwanensis VLB120Δ C Δ ttV for stereospecific epoxidation of toxic styrene alleviates carrier solvent use. Biotechnol. J.:1600558.

1. An isolated nucleic acid fragment encoding an oxygenase subunit (StyA) and an isolated nucleic acid fragment encoding a reductase subunit (StyB), wherein the polypeptides encoded for by the nucleotide sequences for the oxygenase subunit (StyA) and the nucleotide sequence for the reductase subunit (StyB) together have activity towards chiral arylsulfides and wherein said nucleic acid fragments are selected from the group
a) nucleic acid fragment comprising a nucleotide sequence of at least 80% sequence identity to SEQ ID NO 1 and a nucleotide sequence of at least 80% sequence identity to SEQ ID NO 2,
b) nucleic acid fragment comprising a sequence complementary to SEQ ID NO 1 and a nucleic acid fragment comprising a sequence complementary to SEQ ID NO 2,
c) nucleic acid fragment comprising a sequence which specifically hybridizes to said nucleic acid fragment of a) or said complementary of b).

2. The isolated nucleic acid fragment of claim 1, wherein the chiral arylsulfide towards which the polypeptides encoded for by the nucleotide sequences for the oxygenase subunit (StyA) and the nucleotide sequence for the reductase subunit (StyB) together have activity, is an arylsulfide of formula I 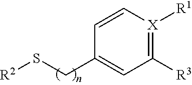
wherein X is C or N, and
wherein if X is N, R₁ is absent
and if X is C, R₁ is selected form the group consisting of a H, NO₂, a halogen, NH₂, an C₁ to C₆ alkyl, or an an C₁ to C₆ O-alkyl, and
wherein n is 0 or 1, and
wherein R² is an C₁ to C₆ alkyl and
wherein R³ is a halogen or H

3. The isolated nucleic acid fragment of claim 2, wherein the chiral arylsulfide towards which the polypeptides encoded for by the nucleotide sequences for the oxygenase subunit (StyA) and the nucleotide sequence for the reductase subunit (StyB) together have activity, is an arylsulfide of formula I 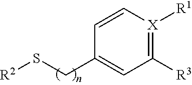
wherein X is N, and
wherein R₁ is absent
and
wherein n is 1, and
wherein R² is an C₁ to C₃ alkyl and
wherein R³ is a halogen or H.

4. A recombinant expression vector comprising the isolated nucleic acid fragments of claim 1, wherein said recombinant expression vector is selected from the group
a) recombinant expression vector comprising the isolated nucleic acid fragments of claim 1 as well as a lac repressor comprising a nucleotide sequence of at least 80% sequence identity to SEQ ID NO 3,
b) recombinant expression vector comprising a nucleotide sequence of at least 80% sequence identity to SEQ ID NO 4
c) recombinant expression vector comprising a sequence complementary to SEQ ID NO 4
d) recombinant expression vector comprising a sequence which specifically hybridizes to said nucleic acid fragment of b) or said complementary of c).

5. The isolated nucleic acid fragments of claim 1, wherein the nucleotide sequence for the oxygenase subunit (StyA) codes for a polypeptide with at least 80% sequence identity to SEQ ID NO 5 and wherein the nucleotide sequence for the reductase subunit (StyB) codes for a polypeptide with at least 80% sequence identity to SEQ ID NO 6.

6. The isolated nucleic acid fragments of claim 1, wherein the nucleotide sequence for the oxygenase subunit (StyA) codes for the polypeptide of SEQ ID NO 5 and wherein the nucleotide sequence for the reductase subunit (StyB) codes for the polypeptide of SEQ ID NO 6.

7. A product comprising the oxygenase subunit (StyA) that codes for a polypeptide with at least 80% sequence identity to SEQ ID NO 5 together with the reductase subunit (StyB) that codes for a polypeptide with at least 80% sequence identity to SEQ ID NO 6 or
the oxygenase subunit (StyA) that codes for a polypeptide with at least 80% sequence identity to SEQ ID NO 5 together with a reductase subunit (StyB) that codes for the polypeptide of SEQ ID NO 6 or
a oxygenase subunit (StyA) that codes for the polypeptide of SEQ ID NO 5 together with the reductase subunit (StyB) that codes for a polypeptide with at least 80% sequence identity to SEQ ID NO 6 or
the oxygenase subunit (StyA) that codes for the polypeptide of SEQ ID NO 5 together with the reductase subunit (StyB) that codes for the polypeptide of SEQ ID NO 6
for the enantioselective oxidation of a compound according to formula I 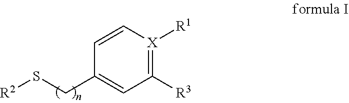
wherein X is C or N, and
wherein if X is N, R₁ is absent
and if X is C, R₁ is selected form the group consisting of a H, NO₂, a halogen, NH₂, an C₁ to C₆ alkyl, or an an C₁ to C₆ O-alkyl, and
wherein n is 0 or 1, and
wherein R² is an C₁ to C₆ alkyl and
wherein R³ is a halogen or H
into the S-sulfynil-enantiomer.

8. Product according to claim 7 for the enantioselective oxidation of 2-chloro-4-(methylsulfanylmethyl)pyridine to 2-Chloro-4-((methyl-S-sulfinyl)methyl) pyridine.

9. A method for enantioselective oxidation of a compound according to formula I 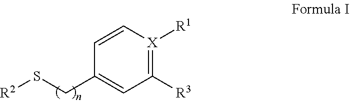
into a S-sulfynil-enantiomer comprising
providing a compound according to formula I,
providing the oxygenase subunit (StyA) that codes for a polypeptide with at least 80% sequence identity to SEQ ID NO 5 together with the reductase subunit (StyB) that codes for a polypeptide with at least 80% sequence identity to SEQ ID NO 6 as polypeptide
reacting said compound according to formula I with said polypeptide for 2-48 hours, with a pH in a range of 5-9 and the temperature is in a range of 15-40° C.

10. The method according to claim 9, wherein the oxygenase subunit (StyA) and the reductase subunit (StyB) are expressed in recombinant cells provided in a cell culture medium, the compound according to formula I is directly added to the cell culture medium once the cells have reached a predetermined cell density and wherein product accumulates in the cell culture medium.

11. The method according to claim 10 wherein the compound according to formula I is added continuously or at regular time intervals at a concentration which is below the conversion rate of the polypeptide.

12. Method according to claim 10 to provide the S-enantiomer of the oxidized compound according to formula I with an ee-value of more than 95%.

13. Method according to claim 10 to provide the S-enantiomer of the oxidized compound according to formula I at a rate of 10 to 60 g/l×h.


SEQUENCE LISTING (6 sequences)

SEQ ID NO: 1 - DNA, 1244 bp, Acinetobacter baylyi
tccatatgcgtcgtatagcaattgttggagcgggtcagtctggattacagctcggtttaagcctgttagacacaggttatgatgtcacaattgtgaccaaccgtaccgcagaccaaattcgtcagggtaaggtcatgtcaagtcagtgtatgtttcataccgctttgcaaactgaacgtgatgttgggctcaacttctgggaagagcaatgtcccgctgttgaaggaattggatttaccctggttagtccagagacaggaaaacctgcattttcgtggagtgcacgtcttgagcgttatgctcaatcggtggatcaacgcgtaaaaatgccttactggattgaagagtttgaacgtcgcggtggcaaactgattattcaggatgttgggattgatgaactagaacaactgactaccgagtatgaactggtgttgctggcagcaggtaagggcgaagtggtgaaacagtttgtgcgtgatgatgagcgcagcacattcgataagccacagcgtgcgcttgctttgacttatgtcacagggatgaaaccgatgtcgccgtattcacgggtgacctttaatgtgattccgggcgttggcgagtacttttgtttccctgcactcaccgtgacaggcccatgcgaaattatggtgttcgaagggattccaggtgggccaatggactgctggcaagatgccaaaacgcctgagcaacatttgcaaatgagtaaagacattctcaatacctatctgccttgggaagctgagcgttgtgaaaatattgaaatcaccgatgcaggcggctatttggctggacgcttcccaccgagcgtgcgtaaaccgatactgacgctaccatctggtcgtcaggtgtttggtatggcagatgcgctggtcgtgaatgatccgattacgggtcaaggttcaaacaacgccgccaaatgcagcaagatttattttgatgccattttagcgcatgacacgcagtcttttacgcctgaatggatgcaacaaacctttgaacgttactggtcttatgccgaaaaagtggtggcttggaccaacagcttactggttccacctcagccacagatgattgatgtattggccgcagcaagccaaaaccaagccattgcctccacgattgccaataactttgatgaccctcgtaatttctctccgtggtggtttgatgcagagcaggcacagcattttatcgaatcgaaaagttgtcagaaagtggcttaa

SEQ ID NO: 2 - DNA, 537 bp, Acinetobacter baylyi
atgaatattaatacatcacatgagctcggtttaaagccgattgatacagaaaatccgagagaaatccgaaatttacttggacagtttgcaactggcgtaaccgtgattaccacgcgtggtcgtgatggacgaaaaatcggaatgaccgccaattcattttcatcattatcacttgatccacccttaattttgtggagtttgtcaaaaactgcaccgagtctgccagactttactgaggcggaatatttcgcgattcacatgctggctcaagagcatcattcactttctggacattttgcacggggttcagaagacaaattcgccagtattgcacatcgtgaatgtgaacgtggcctacctttgcttgaagatgtacttgcgacattggtgtgtaaaaacattaaccaatatgaaggcggtgatcacctgatttttatcggtcagattgagcattatcaacaacgcatcggtgagccattggtttttcatgcgggtaaatatcgtattgcagcagagcatccagagctcagtgcataa

SEQ ID NO: 3 - DNA, 1083 bp, Artificial Sequence
  lacI gene coding for lac repressor
tcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcac

SEQ ID NO: 4 - DNA, 8067 bp, Artificial Sequence
  DNA sequence of the plasmid pCW077_pStyAB_ADP1_lac
cgacctgcagccaagcttctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatgcagcctgaaaggcaggccgggccgtggtggccacggcctctaggccagatccagcggcatctgggttagtcgagcgcgggccgcttcccatgtctcaccagggcgagcctgtttcgcgatctcagcatctgaaatcttcccggccttgcgcttcgctggggccttacccaccgccttggcgggcttcttcggtccaaaactgaacaacagatgtgtgaccttgcgcccggtctttcgctgcgcccactccacctgtagcgggctgtgctcgttgatctgcgtcacggctggatcaagcactcgcaacttgaagtccttgatcgagggataccggccttccagttgaaaccactttcgcagctggtcaatttctatttcgcgctggccgatgctgtcccattgcatgagcagctcgtaaagcctgatcgcgtgggtgctgtccatcttggccacgtcagccaaggcgtatttggtgaactgtttggtgagttccgtcaggtacggcagcatgtctttggtgaacctgagttctacacggccctcaccctcccggtagatgattgtttgcacccagccggtaatcatcacactcggtcttttccccttgccattgggctcttgggttaaccggacttcccgccgtttcaggcgcagggccgcttctttgagctggttgtaggaagattcgatagggacacccgccatcgtcgctatgtcctccgccgtcactgaatacatcacttcatcggtgacaggctcgctcctcttcacctggctaatacaggccagaacgatccgctgttcctgaacactgaggcgatacgcggcctcgaccagggcattgcttttgtaaaccattgggggtgaggccacgttcgacattccttgtgtataaggggacactgtatctgcgtcccacaatacaacaaatccgtccctttacaacaacaaatccgtcccttcttaacaacaaatccgtcccttaatggcaacaaatccgtccctttttaaactctacaggccacggattacgtggcctgtagacgtcctaaaaggtttaaaagggaaaaggaagaaaagggtggaaacgcaaaaaacgcaccactacgtggccccgttggggccgcatttgtgcccctgaaggggcgggggaggcgtctgggcaatccccgttttaccagtcccctatcgccgcctgagagggcgcaggaagcgagtaatcagggtatcgaggcggattcacccttggcgtccaaccagcggcaccagcggcgcctgagaggcgaattgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaatcgatgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaatcgattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcataggggatctccaatcgtgccttggcgcagcgacagccctcggtcccccagatagccattgatcctctctcgcctgtcccctcagttcagtaatttcctgcatttgcctgtttccagtcggtagatattccacaaaacagcagggaagcagcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagctcgagtatacttttcactatatcacttaatgccgattattttctagaaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgtcgtcgccgcacttatgactgtcttctttatcatgcaactcgtaggacaggtgccggcagcgcccaacagtcccccggccacggggcctgtctcggtcgatcattcagcccggctcatagatatgcgggcagtgagcgcaacgcaattaatgtaagttagctcactcattaggcaccccaggcttgacactttatgcttccggctcgtataatgtgtggaattgtgagcggataacaataacaatttcacacaggatctaggaaccagtactggagaattccatatgcgtcgtatagcaattgttggagcgggtcagtctggattacagctcggtttaagcctgttagacacaggttatgatgtcacaattgtgaccaaccgtaccgcagaccaaattcgtcagggtaaggtcatgtcaagtcagtgtatgtttcataccgctttgcaaactgaacgtgatgttgggctcaacttctgggaagagcaatgtcccgctgttgaaggaattggatttaccctggttagtccagagacaggaaaacctgcattttcgtggagtgcacgtcttgagcgttatgctcaatcggtggatcaacgcgtaaaaatgccttactggattgaagagtttgaacgtcgcggtggcaaactgattattcaggatgttgggattgatgaactagaacaactgactaccgagtatgaactggtgttgctggcagcaggtaagggcgaagtggtgaaacagtttgtgcgtgatgatgagcgcagcacattcgataagccacagcgtgcgcttgctttgacttatgtcacagggatgaaaccgatgtcgccgtattcacgggtgacctttaatgtgattccgggcgttggcgagtacttttgtttccctgcactcaccgtgacaggcccatgcgaaattatggtgttcgaagggattccaggtgggccaatggactgctggcaagatgccaaaacgcctgagcaacatttgcaaatgagtaaagacattctcaatacctatctgccttgggaagctgagcgttgtgaaaatattgaaatcaccgatgcaggcggctatttggctggacgcttcccaccgagcgtgcgtaaaccgatactgacgctaccatctggtcgtcaggtgtttggtatggcagatgcgctggtcgtgaatgatccgattacgggtcaaggttcaaacaacgccgccaaatgcagcaagatttattttgatgccattttagcgcatgacacgcagtcttttacgcctgaatggatgcaacaaacctttgaacgttactggtcttatgccgaaaaagtggtggcttggaccaacagcttactggttccacctcagccacagatgattgatgtattggccgcagcaagccaaaaccaagccattgcctccacgattgccaataactttgatgaccctcgtaatttctctccgtggtggtttgatgcagagcaggcacagcattttatcgaatcgaaaagttgtcagaaagtggcttaagcggccgcacttaagttacgcgtggataggagatatcatatgaatattaatacatcacatgagctcggtttaaagccgattgatacagaaaatccgagagaaatccgaaatttacttggacagtttgcaactggcgtaaccgtgattaccacgcgtggtcgtgatggacgaaaaatcggaatgaccgccaattcattttcatcattatcacttgatccacccttaattttgtggagtttgtcaaaaactgcaccgagtctgccagactttactgaggcggaatatttcgcgattcacatgctggctcaagagcatcattcactttctggacattttgcacggggttcagaagacaaattcgccagtattgcacatcgtgaatgtgaacgtggcctacctttgcttgaagatgtacttgcgacattggtgtgtaaaaacattaaccaatatgaaggcggtgatcacctgatttttatcggtcagattgagcattatcaacaacgcatcggtgagccattggtttttcatgcgggtaaatatcgtattgcagcagagcatccagagctcagtgcataacatatgcttggcgcgcccgggatccgt

SEQ ID NO: 5 - protein, 412 aa, Acinetobacter baylyi
MRRIAIVGAGQSGLQLGLSLLDTGYDVTIVTNRTADQIRQGKVMSSQCMFHTALQTERDVGLNFWEEQCPAVEGIGFTLVSPETGKPAFSWSARLERYAQSVDQRVKMPYWIEEFERRGGKLIIQDVGIDELEQLTTEYELVLLAAGKGEVVKQFVRDDERSTFDKPQRALALTYVTGMKPMSPYSRVTFNVIPGVGEYFCFPALTVTGPCEIMVFEGIPGGPMDCWQDAKTPEQHLQMSKDILNTYLPWEAERCENIEITDAGGYLAGRFPPSVRKPILTLPSGRQVFGMADALVVNDPITGQGSNNAAKCSKIYFDAILAHDTQSFTPEWMQQTFERYWSYAEKVVAWTNSLLVPPQPQMIDVLAAASQNQAIASTIANNFDDPRNFSPWWFDAEQAQHFIESKSCQKVA

SEQ ID NO: 6 - protein, 178 aa, Acinetobacter baylyi
MNINTSHELGLKPIDTENPREIRNLLGQFATGVTVITTRGRDGRKIGMTANSFSSLSLDPPLILWSLSKTAPSLPDFTEAEYFAIHMLAQEHHSLSGHFARGSEDKFASIAHRECERGLPLLEDVLATLVCKNINQYEGGDHLIFIGQIEHYQQRIGEPLVFHAGKYRIAAEHPELSA